(19) United States
(12) Patent Application Publication
(10) Pub. No.: US 2010/0152858 A1
(43) Pub. Date: Jun. 17, 2010

(54) Structure improvement of orthopaedic implant

(75) Inventors: Cheng-Kuang Lu, Hsinchu (TW); Yu-Liang Liu, Hsinchu (TW); Jiann-Jong Liau, Hsinchu (TW)

Correspondence Address: BACON & THOMAS, PLLC, 625 SLATERS LANE, FOURTH FLOOR, ALEXANDRIA, VA, 22314-1176, US

(73) Assignee: UNITED ORTHOPEDIC CORP., Hsinchu (TW)

(21) Appl. No.: 12/591,435

(22) Filed: Nov. 19, 2009

(30) Foreign Application Priority Data
Dec. 17, 2008 (TW) 097222604

(51) Int. Cl. A61F 2/38

(52) U.S. Cl. 623/20.32

(57) ABSTRACT

A structure improvement of orthopaedic implant includes a tibial baseplate, a tibial insert, and a reinforcement. The tibial baseplate forms a recess having a bottom that has a central portion defining a through hole extending through the tibial baseplate. The through hole has a top circumference that defines a circumferential groove extending outward. The tibial insert has a bottom forming a projection corresponding to the recess of the tibial baseplate for press-fitting to the tibial baseplate. A support is formed on at a central portion of a top of the tibial insert and defines a bore that extends through the tibial insert. The tibial insert forms two curved surfaces on opposite sides of the support to support contact and rolling. The reinforcement is inset in the tibial insert and includes a sleeve and a bolt.

### FIELD OF THE INVENTION

The present invention relates to a structure improvement of orthopaedic implant, and in particular to an orthopaedic implant comprising a special design of reinforcement to enhance stable coupling between a tibial insert and a tibial baseplate to offer the advantages of resistance against external force, prevention of deformation, and extension of lifespan, and to be applicable to severe bone damage or second knee replacement surgery.

### BACKGROUND OF THE INVENTION

Due to aging, joints of human beings may suffer wear or degradation caused by long term use or degeneration, leading to pain or incapability of providing proper moving functions, as a consequence of which daily living is affected. When a joint suffers severe pathological changes, artificial knee joint replacement provides a measure to release the pain and improve joint function. Artificial knee joint is often composed of components made of metal or plastics and can be fixed to bones to replace the original knee joint to restore the normal function of the knee joint.

Artificial knee joint replacement is a common surgical procedure to treat degradation or lesion of knee joint. A typical artificial knee joint comprises a femural implant, a tibial baseplate, a tibial insert, and a patellar implant. The tibial baseplate is fixed to a surface of a tibia of a patient, and the tibial insert is coupled to the tibial baseplate through mechanic measures. The stability of the knee joint heavily depends on the arrangements between femural implant and tibial insert and between tibial inset and the tibial baseplate. The tibial insert forms thereon a support that constrains the movement between the femural implant and the tibial insert, but it is often made of plastics and is susceptible to fracture caused by long resistance against external forces acting thereon. Daily activity of human beings causes loads and shear forces acting on the artificial knee joint, especially the tibial insert and this may cause dislocation between the tibial insert and the tibial baseplate, or may even separate the tibial insert from the tibial baseplate. Thus, the support formed on the tibial insert must be of sufficient mechanical strength and proper coupling between the tibial insert and the tibial baseplate is important and necessary.

An L-shaped reinforcement inset in the tibial insert is a common way to improve the mechanical strength of the support formed on the tibial insert. Since a metal object is inset in the support of the tibial insert, the mechanical strength of the support for resistance against external forces is increased. However, this arrangement only improves the stability between the tibial insert and the femural implant. One common way for coupling the tibial insert and the tibial baseplate is using a bolt to joint the two components. This only improves the coupling between the two components, but does not increase the capability of resisting shear force. Further, the bolt and the tibial insert are separate parts, which are combined together only when they are put into use. This lowers the efficiency of operation.

Thus, a good design of artificial knee joint must offer: (1) sufficient stability for movement of the knee joint for reducing unnecessary activity or dislocation of joint, (2) sufficient strength for resisting external forces, (3) protection against excessive wear of implants for extension of lifespan of the implants, and (4) improved operation efficiency for reducing the time required for surgical operations.

In view of the above drawbacks, the present invention aims to provide a novel design of orthopaedic implant that overcomes the above problems.

### SUMMARY OF THE INVENTION

An objective of the present invention is to provide a structure improvement of orthopaedic implant, which comprises a reinforcement inset in a tibial insert and coupled, in a downward direction, to a stem or an offset stem adaptor to drive a sleeve of the reinforcement to project beyond a bottom of the tibial insert for fitting into a groove defined in a tibial baseplate to provide the efficacies of stable coupling, resistance against external forces, prevention of deformation, and extension of lifespan thereby enhancing practicability and inventiveness of the present invention.

Another objective of the present invention is to provide a structure improvement of orthopaedic implant, wherein a reinforcement is directly set inside a bore defined in a tibial insert, which shows a configuration having an expanded upper section and a reduced lower section, and a sleeve of the reinforcement has a circumferential outer surface that forms threading or is made roughened, thereby providing the efficacies of increasing operation efficiency, providing safety of use, and making a stable coupling and thus enhancing the inventiveness and safety of the present invention.

A further objective of the present invention is to provide a structure improvement of orthopaedic implant, wherein the components of the orthopaedic implant are of modular designs and suit for difference of individuals and allow for partial replacement to thereby offer the efficacies of being easy to use and reducing costs and thus enhancing the practicability and convenience of the present invention.

To achieve the above objectives, the present invention provides a structure improvement of orthopaedic implant, which comprises a tibial baseplate, a tibial insert, and a reinforcement. The tibial baseplate forms a recess having a bottom that has a central portion defining a through hole extending through the tibial baseplate. The through hole has a top circumference that defines a circumferential groove extending outward. The recess has a side wall defining a retention slot. The tibial insert has a bottom forming a projection corresponding to the recess of the tibial baseplate for press-fitting to the tibial baseplate. A retention pawl is formed on one side of the projection and is engageable with the retention slot of the tibial baseplate. A support is formed on at a central portion of a top of the tibial insert and defines a bore that extends through the tibial insert. The tibial insert forms two curved surfaces on opposite sides of the support to support contact and rolling. The reinforcement is inset in the tibial insert and comprises a sleeve and a bolt. The sleeve is fit in the bore of the tibial insert, and the sleeve forms a bore having a bottom opening around which a stop is formed. The bolt is received in the sleeve in such a way that a shank of the bolt projects outward beyond the bore of the sleeve and a head of the bolt is supported on the stop. As such, the advantages of stable coupling, resistance against stress, prevention of deformation, extension of lifespan, increasing safety of use are provided so as to realize practicability, inventiveness, safety, and convenience.

### BRIEF DESCRIPTION OF THE DRAWINGS

The present invention will be apparent to those skilled in the art by reading the following description of a preferred embodiment thereof with reference to the drawings, in which:

FIG. 1 a perspective view of an orthopaedic implant constructed in accordance with the present invention;

FIG. 2 an exploded view of the orthopaedic implant constructed in accordance with the present invention;

FIG. 3 a cross-sectional view of the orthopaedic implant constructed in accordance with the present invention;

FIG. 4 is a schematic view showing an assembled form of the orthopaedic implant of the present invention; and

FIG. 5 is a schematic view showing a use of the orthopaedic implant of the present invention.

### DETAILED DESCRIPTION OF THE PREFERRED EMBODIMENT

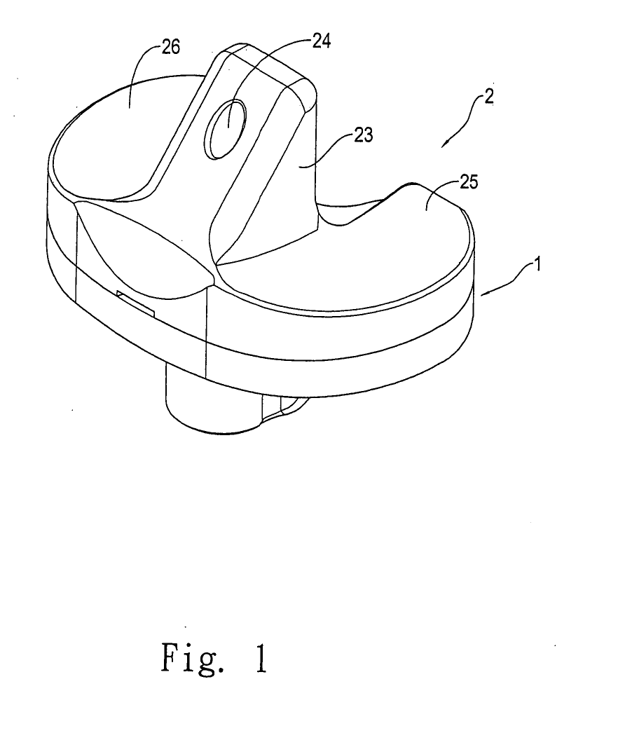

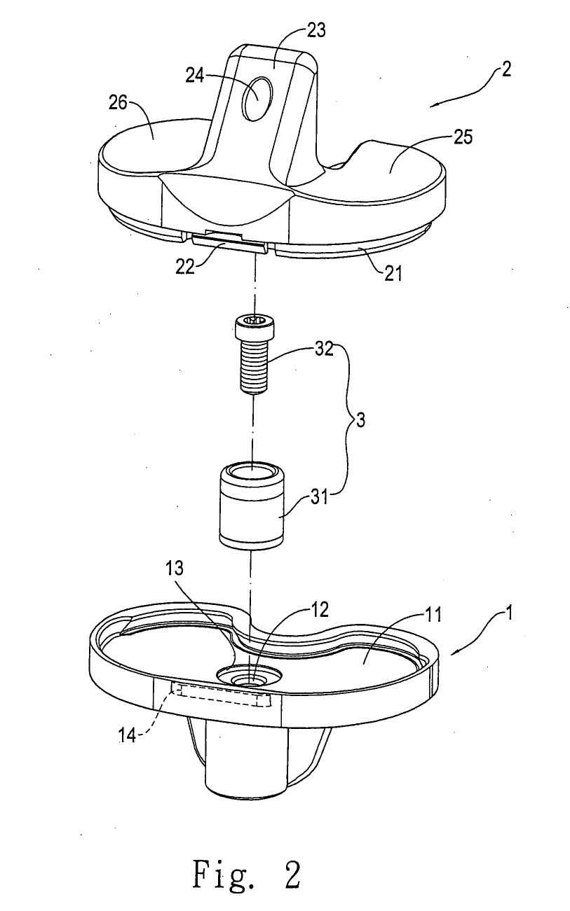

With reference to the drawings and in particular to FIGS. 1-3, which show, respectively, a perspective view, an exploded view, and a cross-sectional view of an orthopaedic implant constructed in accordance with the present invention, the orthopaedic implant of the present invention comprises a tibial baseplate 1, a tibial insert 2, and a reinforcement 3.

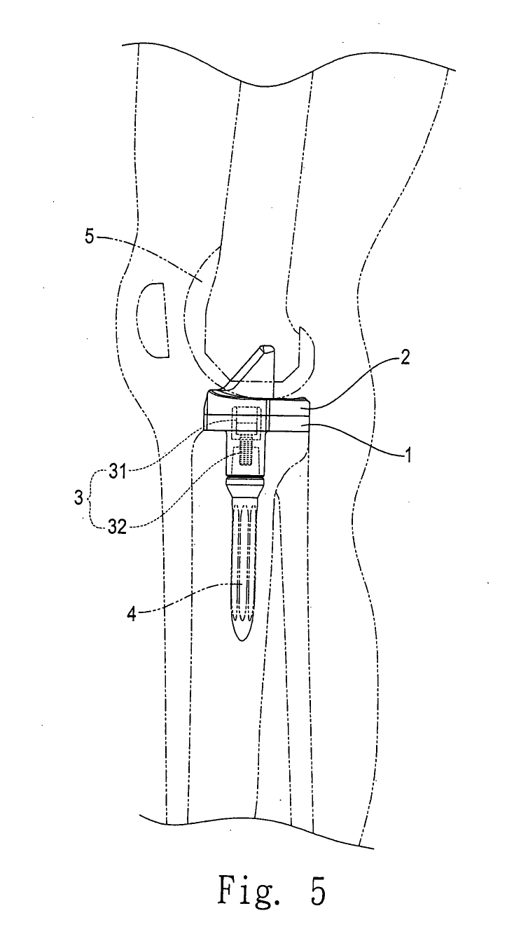

The tibial baseplate 1 is of a modular design having various sizes and forms a recess 11. The recess 11 has a bottom that defines, in a central portion thereof, a through hole 12 extending through the tibial baseplate 1. The through hole 12 has a top circumference that defines a circumferential groove 13 extending outward. The lower portion of the through hole 12 is coupled to a stem 4 (see FIG. 5), or an offset adaptor (not shown). The through hole 12 and the stem 4 or the offset adaptor have ends that are of inclination according to Morse taper. A side wall of the recess 11 defines a retention slot 14 for retaining engagement.

The tibial insert 2 is of a modular design of various sizes and has a bottom forming a projection 21 corresponding to the recess 11 of the tibial baseplate 1 for coupling to, such as press-fitting, the tibial baseplate 1. (In the embodiment illustrated, the side wall of the recess 11 of the tibial baseplate 1 is made in the form of a dovetail and consequently, a side surface of the projection 21 is also made dovetailed.) A retention pawl 22 is formed on one side of the projection 21 and the retention pawl 22 is engageable with the retention slot 14 of the tibial baseplate 1. A support 23 is formed on a top surface of the tibial insert 2, substantially at a central portion thereof, for extending into a femural implant 5 (see FIG. 5) to provide relative movement of the femural implant 5 on the tibial insert 2. The support 23 forms a bore 24 that extends through the tibial insert 2. The bore 24 is of a configuration having a reduced upper section and an expanded lower section. The tibial insert 2 forms, on the top thereof, two curved surfaces 25, 26 on opposite sides of the support 23 to support contact and rolling of the femural implant 5 thereon. (The two curved surfaces 25, 26 are smooth arc surfaces for reducing stress concentration and wear of the tibial insert 2.)

The reinforcement 3 is inset in the tibial insert 2. The reinforcement 3 is composed of a sleeve 31 and a bolt 32. The sleeve 31 is fit in the bore 24 of the tibial insert 2, specifically in the expanded lower section thereof. (In the embodiment illustrated, the sleeve 31 has an outer circumferential surface that is provided with thread or is made roughened (not shown) to facilitate tight engagement between the sleeve 31 and the tibial insert 2.) The sleeve 31 forms a bore 311 having a bottom opening around which a stop 312 is formed. The bolt 32 is received in the bore 311 of the sleeve 31 in such a way that a shank 322 projects outward beyond the bore 311 and a head 321 is supported on the stop 312. Since the bore 24 is of an upper-section-reduced and lower-section-expanded configuration, when the bolt 32 breaks or loosens, the bolt 32 is not allowed to get out of the tibial insert 2 to cause damage or infection to the patient.

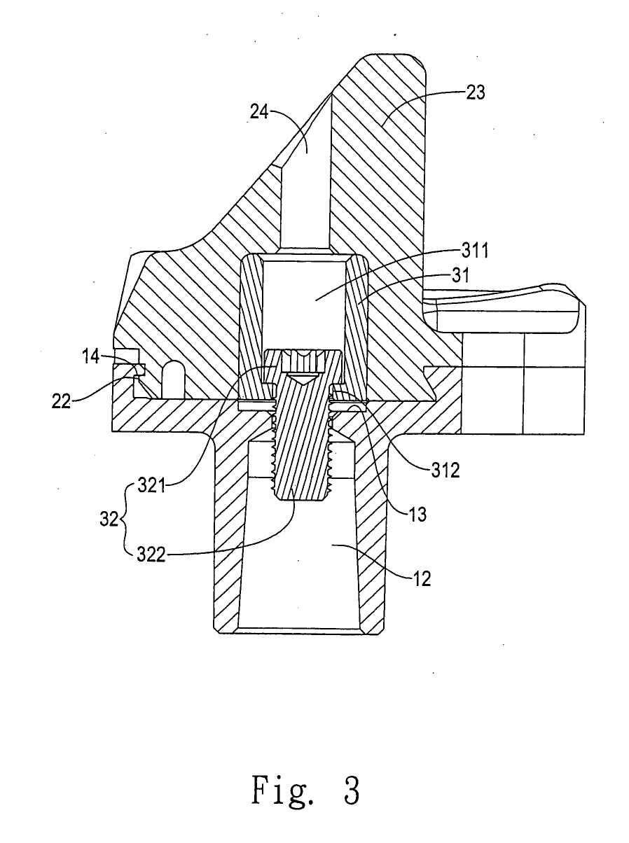

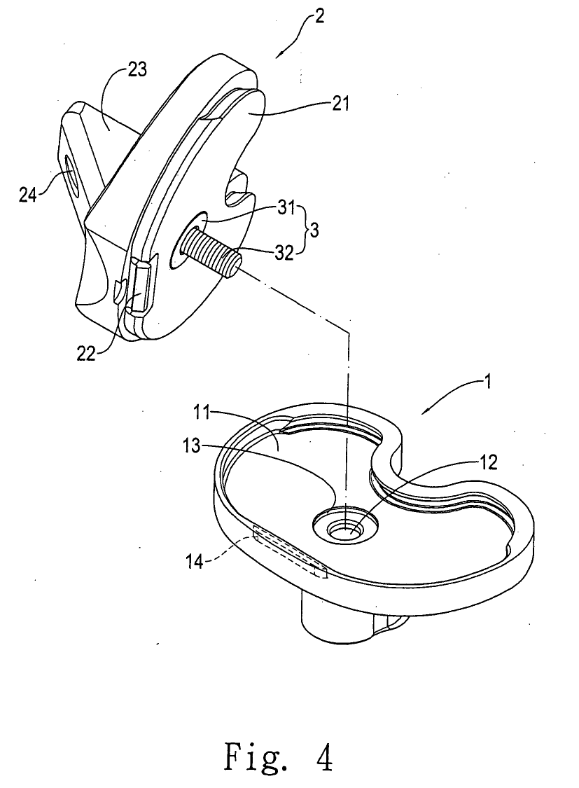

Referring to FIGS. 4 and 5, which are schematic views respectively showing an assembled form of the present invention and the use of the present invention, in use, the tibial insert 2 is press-fit to the tibial baseplate 1. (In the embodiment illustrated, after the formation of the tibial insert 2, the reinforcement 3 is already inset in the tibial insert 2.) Since the projection 21 of the tibial insert 2 is formed to correspond to the recess 11 of the tibial baseplate 1, they can be tightly and efficiently coupled to each other and with the retention pawl 22 on one side of the tibial insert 2 engaging the retention slot 14 of the tibial baseplate 1, a more secured and more stable coupling between the tibial insert 2 and the tibial baseplate 1 can be realized to prevent undesired separation and to improve safety of use. Further, the lower portion of the through hole 12 of the tibial baseplate 1 is coupled to the stem 4 and the inclination of the through hole 12 and the stem 4 is set in the form of Morse Taper, so that easy mounting and dismounting can be realized therebetween. (The stem 4 that is shown in the instant embodiment can be instead replaced by an offset adaptor.) An end of the stem 4 forms a coupling hole (not shown) inside which inner thread is formed to allow inter-engagement between the coupling hole and the bolt 32 of the reinforcement 3. A tool, such as a screwdriver, may be inserted into the sleeve 31 that is fit in the bore 24 of the tibial insert 2 to rotate the bolt 32 and the stem 4 and at the same time also drive the sleeve 31 to project beyond an end of the tibial insert 2 to fit into the groove 13 of the tibial baseplate 1 for improving resistance against shear force after they are coupled. Further, the support 23 that is formed on the central portion of the top of the tibial insert 2 is inserted into the femural implant 5 to provide contact and rolling of the femural implant 5 on the curved surfaces 25, 26 of the tibial insert 2. Since the curved surfaces 25, 26 of the tibial insert 2 are smooth arc surfaces, when the femural implant 5 rolls on the curved surfaces 25, 26 of the tibial insert 2, the knee joint is allowed to undergo smooth bending and stretching movements with reduced stress concentration and wear of the tibial insert 2. The reinforcement 3 of the present invention (composed of a sleeve 31 and a bolt 32) that is inset in the tibial insert 2 helps reinforcing the tibial insert 2 to resist stress induced in the tibial insert 2 by the movement of the femural implant 5 thereby realizing protection against damage and deformation caused thereby and extension of the service life. Further, the bolt 32 of the reinforcement 3 is coupled to the stem 4 to make the sleeve 31 fit into the groove 13 for improving resistance against shear force. Further, due to the unique configuration of the bore 24 (as shown in FIG. 3 that provides an upper-section-reduced and lower-section-expanded bore 24), when the bolt 32 of the reinforcement 3 breaks or loosens, the bolt 32 is not allowed to get out of the tibial insert 2 to cause damage or infection and also offering safety of use. In addition, the arrangement that sets the reinforcement 3 inside the tibial insert 2 helps improving efficiency of operation.

The present invention provides an orthopaedic implant that has the following advantages. A reinforcement 3 is inset in a tibial insert 2 and the reinforcement 3 comprises a bolt 32 that is coupled, in a downward direction, with a stem 4 or an offset adaptor of a stem 4, so as to allow a sleeve 31 of the reinforcement 3 to project beyond a bottom of the tibial insert 2 to fit into a circumferential groove defined in a tibial baseplate 1, thereby offering the efficacies of stable coupling, resistance against external forces, prevention of deformation and extension of lifespan and thus enhancing practicability and inventiveness of the present invention. The reinforcement 3 is directly set inside a bore 24 of the tibial insert 2, which shows a configuration having an expanded upper section and a reduced lower section, and a sleeve 31 of the reinforcement 3 has a circumferential outer surface that forms threading or is made roughened, thereby providing the efficacies of increasing operation efficiency, providing safety of use, and making a stable coupling and thus enhancing the inventiveness and safety of the present invention. The components of the present invention are of modular designs and suit for difference of individuals and allow for partial replacement to thereby offer the efficacies of being easy to use and reducing costs and thus enhancing the practicability and convenience of the present invention. To conclude, the orthopaedic implant in accordance with the present invention provides excellent practicability, inventiveness, safety, and convenience.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. a structure improvement of orthopaedic implant, comprising:
a tibial baseplate, which forms a recess having a bottom that has a central portion defining a through hole extending through the tibial baseplate, the through hole having a top circumference that defines a circumferential groove extending outward, the recess having a side wall defining a retention slot;
a tibial insert, which has a bottom forming a projection corresponding to the recess of the tibial baseplate for press-fitting to the tibial baseplate, a retention pawl being formed on one side of the projection and engageable with the retention slot of the tibial baseplate, a support being formed on at a central portion of a top of the tibial insert and forming a bore that extends through the tibial insert, the tibial insert forming two curved surfaces on opposite sides of the support to support contact and rolling; and
a reinforcement, which is inset in the tibial insert and comprises a sleeve and a bolt, the sleeve being fit in the bore of the tibial insert, the sleeve forming a bore having a bottom opening around which a stop is formed, the bolt being received in the sleeve in such a way that a shank of the bolt projects outward beyond the bore of the sleeve and a head of the bolt is supported on the stop.

2. The structure improvement of orthopaedic implant as claimed in claim 1, wherein the tibial baseplate is coupled to a stem or an offset adaptor of stem with an end of the stem or the offset adaptor fit into the through hole of the tibial baseplate.

3. The structure improvement of orthopaedic implant as claimed in claim 2, wherein an end of the stem or the offset adaptor forms a coupling hole inside which inner thread is formed to allow inter-engagement between the coupling hole and the bolt of the reinforcement.

4. The structure improvement of orthopaedic implant as claimed in claim 3, wherein the bolt of the reinforcement is in threading engagement with the stem or the offset adaptor to drive the sleeve of the reinforcement to project beyond an end of the tibial insert for fitting into the groove of the tibial baseplate.

5. The structure improvement of orthopaedic implant as claimed in claim 1, wherein the sleeve of the reinforcement has an outer circumferential surface that forms a thread or is made roughened to improve tight engagement between the sleeve and the tibial insert.

6. The structure improvement of orthopaedic implant as claimed in claim 1, wherein the bore of the tibial insert has a configuration that has a reduced upper section and an expanded lower section to help preventing the detached bolt fall from the tibial insert.

7. The structure improvement of orthopaedic implant as claimed in claim 1, wherein the reinforcement is directly inset in the tibial insert.
